SEQUENCE LISTING (38 sequences)

SEQ ID NO: 1 - DNA, 18 bp, Artificial
  synthetic oligonucleotide forward primer
cggtgcgcgtcgctcagg

SEQ ID NO: 2 - DNA, 18 bp, Artificial
  synthetic oligonucleotide reverse primer
tccgatatcccgggtcgt

SEQ ID NO: 3 - DNA, 23 bp, Homo sapiens
caactttcggatgataaccagta

SEQ ID NO: 4 - DNA, 24 bp, Homo sapiens
agaattttaccagatctccaatgt

SEQ ID NO: 5 - DNA, 27 bp, Homo sapiens
cacttagagcggatggtaattatgtct

SEQ ID NO: 6 - DNA, 21 bp, Homo sapiens
gagggcaagcctctctatatc

SEQ ID NO: 7 - DNA, 23 bp, Homo sapiens
tccaggggatgcatgtcctgttc

SEQ ID NO: 8 - DNA, 21 bp, Homo sapiens
cctttccctggcctagtacag

SEQ ID NO: 9 - DNA, 24 bp, Homo sapiens
ttcaaccggatgccaactgagcac

SEQ ID NO: 10 - DNA, 19 bp, Homo sapiens
tcctgaagggatgagttcc

SEQ ID NO: 11 - DNA, 20 bp, Homo sapiens
acccggatgcaacagtcacc

SEQ ID NO: 12 - DNA, 21 bp, Homo sapiens
tgcaagaattgagctttaata

SEQ ID NO: 13 - DNA, 27 bp, Homo sapiens
ccttatttaggggatgtacaaacactt

SEQ ID NO: 14 - DNA, 18 bp, Homo sapiens
acgcccggcaagattcat

SEQ ID NO: 15 - DNA, 24 bp, Homo sapiens
agaggagtggatgcctctaatgtt

SEQ ID NO: 16 - DNA, 19 bp, Homo sapiens
ggacacgcagaatgggaga

SEQ ID NO: 17 - DNA, 21 bp, Homo sapiens
atgaaaaggatggagtcactg

SEQ ID NO: 18 - DNA, 25 bp, Homo sapiens
aaatacatctaaccatatttaagag

SEQ ID NO: 19 - DNA, 22 bp, Homo sapiens
aatgaaaaggatggagtcactg

SEQ ID NO: 20 - DNA, 22 bp, Homo sapiens
accccaggaaaggacaaaacaa

SEQ ID NO: 21 - DNA, 21 bp, Homo sapiens
agttcttgggatgaaggaaat

SEQ ID NO: 22 - DNA, 23 bp, Homo sapiens
cattccatgatataatctttgtg

SEQ ID NO: 23 - DNA, 23 bp, Homo sapiens
gttgatgggatggttagaaaaag

SEQ ID NO: 24 - DNA, 21 bp, Homo sapiens
acaacacaaggtagtttcacg

SEQ ID NO: 25 - DNA, 22 bp, Homo sapiens
caggtaggatggggcttgtgta

SEQ ID NO: 26 - DNA, 24 bp, Homo sapiens
tctctaacatacctatcaagtcta

SEQ ID NO: 27 - DNA, 22 bp, Homo sapiens
tcctggggatggaaataaggac

SEQ ID NO: 28 - DNA, 20 bp, Homo sapiens
agcggaaactgccttagctg

SEQ ID NO: 29 - DNA, 22 bp, Homo sapiens
tgctgggatgcattttgatgtt

SEQ ID NO: 30 - DNA, 19 bp, Homo sapiens
cccacacaagggattgaaa

SEQ ID NO: 31 - DNA, 24 bp, Homo sapiens
agtataacaggatggaaagagctg

SEQ ID NO: 32 - DNA, 23 bp, Homo sapiens
attcctattcttgaaacctctgg

SEQ ID NO: 33 - DNA, 21 bp, Homo sapiens
tgcaaatcggatgcctctagc

SEQ ID NO: 34 - DNA, 23 bp, Homo sapiens
ttctacttttattccatcattgc

SEQ ID NO: 35 - DNA, 24 bp, Homo sapiens
ttgagacggatgtgactaacactg

SEQ ID NO: 36 - DNA, 23 bp, Homo sapiens
ccaggtaatgaataatgtgaggt

SEQ ID NO: 37 - DNA, 24 bp, Homo sapiens
agtttacggatgatttaggtctcc

SEQ ID NO: 38 - DNA, 22 bp, Homo sapiens
gcaattgtaagattcagggaag

(19) United States
(12) Patent Application Publication
(10) Pub. No.: US 2005/0214840 A1
(43) Pub. Date: Sep. 29, 2005

(54) Restriction enzyme mediated method of multiplex genotyping

(76) Inventor: Xiangning Chen, Richmond, VA (US)

Correspondence Address: WHITHAM, CURTIS & CHRISTOFFERSON, P.C., 11491 SUNSET HILLS ROAD, SUITE 340, RESTON, VA, 20190, US

(21) Appl. No.: 11/086,401

(22) Filed: Mar. 23, 2005

Related U.S. Application Data
(60) Provisional application No. 60/555,357, filed on Mar. 23, 2004.

(51) Int. Cl. C12Q 1/68; C12P 19/34

(52) U.S. Cl. 435/6; 435/91.2

(57) ABSTRACT

A method for single nucleotide polymorphism (SNP) genotyping using widely available DNA sequencers is provided. A restriction endonuclease recognition site is incorporated into a PCR primer for the SNP, a restriction enzyme is used to cleave the DNA and create extendable ends at target polymorphic sites, and an extension reaction is used to create allele-specific extension products that can be distinguished using DNA sequencers or other detection platforms.

### CROSS-REFERENCE TO RELATED APPLICATIONS

This application claims benefit of U.S. provisional patent application 60/555,357, filed Mar. 23, 2004, the complete contents of which are hereby incorporated by reference.

### DESCRIPTION

### BACKGROUND OF THE INVENTION

1. Field of the Invention

The invention generally relates to a method of genotyping single nucleotide polymorphisms (SNPs) using DNA sequencers. In particular, a restriction endonuclease recognition site is incorporated into a PCR primer for the SNP, a restriction enzyme is used to cleave the DNA and create extendable ends at target polymorphic sites, and an extension reaction is used to create allele-specific extension products that can be distinguished using DNA sequencers.

2. Background of the Invention

Genetic variations are the basis of human diversity and play an important role in human diseases. Single nucleotide polymorphisms (SNPs) are the most abundant variation in the human genome^{1.2}. The large number of SNPs available in the public databases makes fine-mapping disease genes realistic and exciting. However, there are practical problems for such studies, the cost and throughput of SNP typing being amongst the most significant^{3,4,5}. Most methods for SNP typing require dedicated instruments, such as microarray techniques^{6,7}, matrix assisted laser desorption/ionization mass spectrometry^{8,9}, the SNP stream system¹⁰, the TaqMan nuclease assay¹¹, the pyrosequencing¹² and the FP-TDI method¹³. The high cost of dedicated instrumentation makes these methods out of reach for many laboratories.

DNA sequencers are one of the most widely available instruments for biomedical research. The throughput and automation of sequencers have been demonstrated in large scale sequencing projects like the human genome project. DNA sequencers separate and detect DNA fragments by size and fluorescence labeling¹⁴. To use sequencers efficiently and cost-effectively, the key is to find a way to generate a series of products of different sizes and colors because sequencers can separate and identify these products every efficiently. The SNaPshot and SNuPe techniques marketed by Applied Biosystems and Amersham Corporation were the first attempts in this direction. Because of the length limitation of extension primers, neither method was used broadly. Recently, Schouten et al. reported a ligation mediated method to quantify target sequences and potentially to type SNPs¹⁵. In the method, a short probe containing a target specific sequence and a common tail was ligated to a large probe, which was produced by cloning a short target sequence and stuffer sequences of variable length. Ligation products were then amplified with a universal primer set and separated and identified by capillary electrophoresis (CE). One of the weaknesses of this method was the cumbersome cloning procedures required for each target. CE has also been used to separate allele-specific PCR products¹⁶. However, allele-specific PCR is not a robust procedure. It has serious problems in multiplexing and does not work for some SNPs¹⁷. As a result, allele specific PCR is not routinely used for SNP typing.

The prior art has thus far failed to provide straightforward, cost effective methods for SNP genotyping, particularly methods that take advantage of the prevalence of DNA sequencers.

### SUMMARY OF THE INVENTION

It is an object of the invention to provide a method for SNP genotyping. In preferred embodiments, the invention utilizes widely available technology (such as DNA sequencing) to analyze DNA sequences produced by the method. According to the method, a restriction enzyme (RE) recognition site is engineered into one of two PCR primers for an SNP of interest. The PCR product thus contains the RE recognition site and will cleave the PCR product. The position of the RE recognition site is designed so that the cleavage site for the RE is immediately adjacent to the targeted SNP site. Digestion of the PCR products by the corresponding RE creates an overhang end structure at the targeted polymorphic site, the innermost nucleotide of which is the SNP. This overhang structure is then extended with a detectable nucleotide complementary to the SNP. The nucleotide may be a directly detectable differentially labeled nucleotide, or the complementary nucleotide may be part of a differentially labeled allele specific ligation adaptor. In either case, the extension reaction produces a product that is differentially labeled and allele-specific. This allele-specific product is detected using a DNA sequencer, which allows the genotype of the SNP to be determined.

The invention provides a method of determining a genotype of a single nucleotide polymorphism (SNP). The first step of the method is amplifying by polymerase chain reaction (PCR) a nucleotide sequence containing the SNP using a first primer and a second primer. The first primer binds to a first strand of DNA containing the SNP and comprises: sequences identical to a recognition site of a restriction enzyme, and a first DNA sequence homologous to nucleotide sequences immediately adjacent to the SNP. The first primer may include a tail comprising DNA sequences that are not homologous to sequences flanking the SNP. The second primer binds to a second strand of DNA containing the SNP and comprises DNA sequences homologous to nucleotide sequences flanking the SNP on the second strand. The second primer may also include a tail comprising DNA sequences that are not homologous to the nucleotide sequence. According to the method, the step of amplifying produces amplification products that contain the restriction enzyme recognition site and a cleavage site of the restriction enzyme, in which a terminal nucleotide of the cleavage site is one allele of the SNP.

The second step of the method is cleaving the amplification product with the restriction enzyme. The step of cleaving produces a cleaved amplification product with an overhang in which a first nucleotide of the overhang is one allele of the SNP.

The third step of the method is joining a detectable complementary nucleotide to the one allele of the SNP to form an allele specific detectable product.

The fourth step of the method is detecting the allele specific detectable product produced in the joining step.

The fifth step of the method is genotyping the SNP based on output from the detecting step.

In some embodiments of the method, the first primer further comprises a second DNA sequence homologous to nucleotide sequences flanking but not immediately adjacent to the SNP. In this case, the sequences identical to a recognition site of a restriction enzyme are located between the first and second DNA sequences of the first primer.

In preferred embodiments of the invention, the restriction enzyme is a type IIS restriction enzyme, examples of which include but are not limited to BbvI, BceAl, BtgZI and FokI. Depending on the restriction enzyme that is used, the overhang may be a 5′ overhang and the step of joining is then carried out by single base extension with differentially labeled nucleotides, e.g. fluorescent dye-terminator nucleotides. In other embodiments, the overhang is a 3′ overhang and the step ofjoining is carried out by a method such as DNA ligation using allele specific ligation adaptors or DNA hybridization using allele specific hybridization probes.

In one embodiment of the method, the first primer is a forward primer and the second primer is a reverse primer. Alternatively, the primers may be designed so that the second primer is a forward primer and the first primer is a reverse primer.

In a preferred embodiment of the invention, the detecting step is performed using an instrument or technique that is capable of determining the nucleotide sequence of the allele-specific detectable product. Examples of such instruments or techniques include but are not limited to DNA sequencers, microarrays, microbeads, microchips, fluorescence resonance energy transfer, fluorescence polarization, melting temperature analysis, and mass spectrometry. In preferred embodiments, the instrument or technique is a DNA sequencer or mass spectrometry.

### BRIEF DESCRIPTION OF THE DRAWINGS

FIG. 1. Schematic of an exemplary embodiment of the invention using allele-specific single base extension.

FIG. 2. Schematic of an exemplary embodiment of the invention using allele-specific ligation adaptors.

FIG. 3. Comparison of single-marker PCR and five-plex multiplex PCR. (A, top) Single-marker PCRs (Nos. 1-18), see Table 2). The results indicated that the inclusion of a restriction recognition site in the forward PCR primers did not change the specificity of PCR. (B, bottom). Some examples of randomly assembled five-plex PCRs. The combinations of markers used were: A, 2/6/7/9/10; B, 1/3/8/12/18; C, 1/2/3/4/6; D, 4/6/7/9/10; E, 2/9/10/11/13; F, 5/10/12/14/16; G, 2/13/14/15/16; H, 2/3/6/7/9; 1,1/5/7/9/12; J, 2/19/13/15/16; K, 11/13/15/16/17; L, 3/8/11/14/17; M, 1/4/10/13/16; N, 4/9/13/15/16; 0, 1/7/10/15/16; P, 3/4/9/10/16; Q, 3/9/10/17/18; and R, 3/4/6/9/12. The numbers in the combinations referred to the order listed in (A, top). These results demonstrate that our two-domain primer design and two-stage protocol performed well for multiplex.

FIG. 4. Electropherograms showing allele discrimination for marker 7, rs246945 (G/C SNP, fragment size 273 bp). After FokI digestions, SBE was performed with R110-ddGTP and TAMRA-ddCTP and extension products were separated by CE. Products with expected size and allele-specific fluorescence labeling were identified. R110-labelled products were blue peaks (B), represented the C allele; TAMRA-labeled products, green peaks (G) represented the G allele. Red peaks (R) were DNA size standard ILS600. (A) C/C homozygote; (B), heterozygote; (C) G/G homozygote.

FIG. 5. Electropherogram showing marker separation and allele discrimination for a five-plex reaction (combiatnion H: 2/3/6/7/9). F, R110-labeled ddGTP/ddUTP (blue peaks, B); T, TAMRA-labeled ddATP/ddCTP (green peaks, G); I, DNA size standard ILS600 (red peaks, R).

FIG. 6. Genotyping results from a five-plex reaction (combination A). Five SNPs were typed for 44 subjects. After the reactions and purification, samples were separated in SCE9610 sequencer. Genotypes were scored by peak size, peak color, and peak height ratio as described I the text.

FIG. 7. Examples of electropherograms showing marker separation and allele discrinmination for pooled multiplex PCRs. (A) Pooling of two five-plex reactions. Ten markers (1/2/7/10/11/13/15/16/17/18) were clearly separated. (B) Three five-plex reactions were pooled. Fifteen markers (1/2/3/4/6/7/8/9/10/12/13/15/16/18) were separated and genotypes scored. The results show that pooling several multiplex PCRs is an effective way to increase throughput and to reduce cost.

### DETAILED DESCRIPTION OF THE PREFERRED EMBODIMENTS OF THE INVENTION

The present invention provides an improved method for SNP genotyping. The method is especially suited for use with widely available laboratory instruments, such as DNA sequencers. According to the method, a restriction enzyme recognition site is engineered in a first of two PCR primers for an SNP of interest. The restriction enzyme is a type IIS RE which cleaves DNA fragments several nucleotides downstream of its recognition site. The distance between the recognition and cutting sites is characteristic of a type IIS RE. Some of these type IIS REs produce 5′ overhang structures at their cleavage sites, while others produce 3′ overhang structures. In the present invention, REs that produce either a 5′ overhang or a 3′ overhang can be used. In one embodiment, the 5′ overhang structures are used with single base extension to discriminate among alleles of the targeted SNPs; in another embodiment, both the 3′ and 5′ overhang structures can be used with DNA ligation and/or DNA hybridization to discriminate among alleles.

During design of the primer, the recognition sequence is placed in the first primer so that, in the PCR product, the cleavage site of the restriction endonuclease will be located immediately adjacent to the targeted SNP. Thus, when the PCR product is cleaved by the restriction enzyme, the cleavage product has an overhang structure in which the innermost nucleotide is the SNP. Subsequently, the nucleotide that complements the SNP is joined to the overhang structure. This can be accomplished by a variety of techniques, including, for example, by single base extension, by DNA ligation, by DNA hybridization, etc. By differentially labeling the complementary nucleotide, it is possible to render the cleavage product allele-specific. For example, the complementary nucleotide may be labeled directly, as with a fluorescently labeled dye-terminator nucleotide; or the complementary nucleotide may be part of a ligation adaptor sequence that is differentially labeled.

The second of the two PCR primers is designed to bind DNA flanking the SNP at a desired predetermined distance on the opposing strand of DNA. Because the location of binding of the second primer is known, the length of the PCR product (and thus of the RE digestion product) will also be predictable. According to the invention, the length of the PCR product can be varied as warranted (to distinguish between PCR products) by varying the primer sequence in order to vary its binding position along the DNA, resulting in shorter or longer PCR products, as desired. In this manner, it is possible to create PCR products for an SNP of interest (and thus RE digestion products for an SNP of interest) whose length differs from that of another SNP of interest. This is useful, for example, if two or more SNP loci are PCR amplified in the same reaction. By using second primers that bind at different positions, the resulting PCR products of each SNP in the reaction will differ in length and thus be distinguishable from one another. Thus, according to the invention, the size of the PCR product (and thus ultimately the size of the product after digestion with an RE) is controlled by the position of the second primer, once a specific RE enzyme has been selected for use in the overall primer design.

The direct use of REs for SNP typing has been reported^{18,19}. However, in these methods, it is only possible to genotype SNPs that either create or disable a recognition sequence. If a SNP creates a new recognition sequence for a restriction enzyme, a digestion of PCR amplified products with the restriction enzymes used in these techniques will produce two fragments. By identifying the number of fragments (via gel electrophoresis), the genotype of the subject can be identified. Thus, these methods use only those REs whose cleavage sites are within their recognition sequences. In contrast, the present invention utilizes a special group of type II REs (type IIS) whose recognition sites are several bases away from their cutting sites. This reference ²⁰ provides a detailed classification of type II restriction endonucleases. FokI, BbvI, BtgZI and BceAl are examples of type IIS enzymes (Table 1).

TABLE 1
Examples of type IIS restriction endonucleases
that can be used with the REM-SBE technique

| | Recognition sequence |
|---|---|
| Restriction endonucleases | and cutting Site (\hat{ }) |
| BbvI | 5′...GCAGC(N)₈\hat{ }...3′ |
| | 3′...CGTCG(N)₁₂\hat{ }...5′ |
| BceAI | 5′...ACGGC(N)₁₀\hat{ }...3′ |
| | 3′...TGCCG(N)₁₄\hat{ }...5′ |
| BtgZI | 5′...GCGATG(N)₁₀\hat{ }...3′ |
| | 3′...CGCTAC(N)₁₄\hat{ }...5′ |
| FokI | 5′...GGATG(N)₉\hat{ }. . .3′ |
| | 3′...CCTAC(N)₁₃\hat{ }...5′ |

Other examples of restriction enzymes that may be used in the practice of the present invention include but are not limited to EcoP15 I, Eci I, BsmF I, Acu I, Bpm I, Mme I, TspDT I, TspGWI, Taq II, Eco57 I, Eco57M I and Gsu I. Some of these enzymes generate 5′ overhangs, and other generate 3′ overhangs.

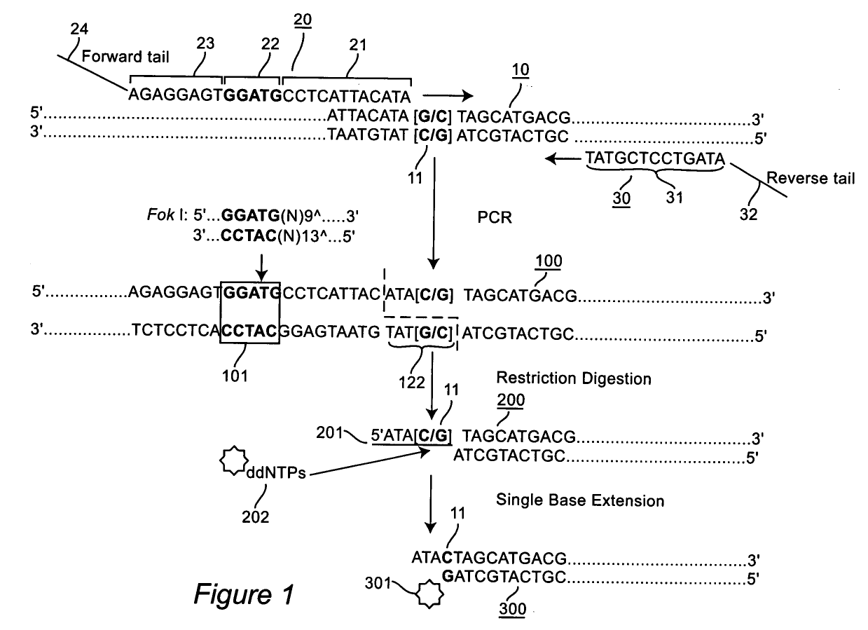

One embodiment of the present invention, as applied to genotyping a locus where the SNP is either G or C, is illustrated schematically in FIG. 1. In the embodiment of the invention depicted in this figure, the primer in which a RE recognition site is engineered is the forward primer, and the primer that controls the length of the PCR product is the reverse primer. However, those of skill in the art will recognize that this need not be the case, i.e. the reverse primer may incorporate the RE recognition sequence, and the forward primer may placed differentially so as to confer length distinctions among SNPs. In FIG. 1, the restriction enzyme is a type II enzyme, FokI.

In the exemplary embodiment of the invention shown in FIG. 1, forward primer 20, reverse primer 30, and nucleotide sequence 10 are depicted. Nucleotide sequence 10 contains SNP 11. Forward primer 20 comprises: a first DNA sequence 21 homologous to the nucleotide sequence 10 immediately adjacent (i.e. the very next nucleotide) and upstream of SNP 11; sequences 22 identical to a recognition site of type II restriction enzyme (RE) FokI; a second DNA sequence 23 homologous to sequences flanking SNP 11 but not immediately adjacent to SNP 11; and a “tail” 24 comprising DNA sequences that are not homologous to the nucleotide sequence 10. Reverse primer 30 comprises DNA sequences 31 homologous to the nucleotide sequence 10 and located downstream of SNP 11, and a “tail” 32 comprising DNA sequences that are not homologous to the nucleotide sequence 10. By SNP we mean “single nucleotide polymorphism”, which is the change of a single nucleotide in a DNA sequence. The change can be a substitution, insertion or deletion. Nomenclature such as “C/G” refers to a substitution in which a cytosine nucleotide (C) may be replaced by a guanine nucleotide (G) in a given sequence context. In genetics terminology, the sequence with the cytosine at the given position is referred to as the “C allele”, that with the guanine nucleotide is referred as the “G allele”. In a homozygous individual, the genotype may be C/C or G/G, i.e. either both Cs or both Gs at the locus on both chromosomes. For a heterozygous individual, the genotype would be C/G, indicating the presence of a C on one chromosome and a G on the other. It should be understood that the depiction in FIG. 1 is exemplary.

PCR amplification of the nucleotide sequence 10 with primers 20 and 30 produces amplification product 100 that contains both RE recognition site 101 and cleavage site 102 of the type II RE, the terminal 5′ nucleotide of the cleavage site being one allele of SNP 11. In other words, the nucleotide of the cleavage site that is furthest away from the recognition site will be one allele of SNP 11. Cleavage site 102 is not sequence dependent, but is determined by which RE will be used to digest the PCR product, which in turn depends on which RE recognition site 22 was engineered into forward primer 20. The RE will simply cut the DNA strand at a position located a characteristic number of nucleotides from its recognition site, regardless of the sequence. Restriction digestion of amplification product 100 with the appropriate type II RE produces a digestion product 200 having a 5′ overhang 201, in which the innermost, 3′ nucleotide of the overhang (i.e. a first nucleotide of the overhang in the 3′ to 5′ direction when a type II RE is used) is an allele of SNP 11. When a single base extension reaction is carried out with digestion product 200 using ddNTPs 202, the single labeled ddNTP 301 that correctly basepairs with the allele of SNP 11 will be added, producing a labeled allele-specific product 300 that can be detected and identified using a DNA sequencer. In FIG. 1, SNP 11 is C and specific ddNTP 301 is G. The length of allele-specific product 300 is known because the length and position of reverse primer 30 (used in the PCR amplification step) is known, and will be different for each SNP. While FIG. 1 illustrates amplification of a C/G SNP, those of skill in the art will recognize that other SNPs (e.g. A/G, A/C, A/T, C/T, G/T, etc.) can also be identified using this technique.

In FIG. 1, primer 20 contains two DNA sequences 21 and 23 both of which are homologous to sequences that flank SNP 11. Sequence 21, which binds immediately adjacent to SNP 11, is included in all first primers 20; however, sequence 23 is preferable, but is not absolutely required in all embodiments of the invention, its chief function being to stabilize the binding of primer 20 to the strand of DNA that includes SNP 11. Those of skill in the art will recognize that such an optional segment would be required when sequence 21 is not sufficient to produce desired specificity and efficiency for PCR. The length of segments 21 and 22 of primer 20 will be determined by the restriction enzyme that is used. For example, with the FokI enzyme, segment 21 will be 9 nucleotides long, and segment 22 will be 5 nucleotides long. With the BtgZI enzyme, segment 21 would be 10 nucleotides long, and segment 22 would be 6 nucleotides long. However, the length of optional segment 23 is variable, and will generally be in the range of 0 to about 45 nucleotides, and preferably in the range of from about 4 to about 30 nucleotides, depending on the sequence that determines the annealing temperature and specificity.

Likewise, the length of homologous segment 31 of second primer 30 may vary from primer to primer, but will generally be in the range of about 8 to about 45 nucleotides, and preferably in the range of from about 15 to about 35 nucleotides, depending on the sequence that determines the annealing temperature and specificity.

Both primer 20 and primer 30 are represented in FIG. 1 as having non-homologous “tail” regions 24 and 32, respectively. The function of the “tail” is to facilitate amplification of multiple SNPs in a single PCR. Thus, the presence of the tail is preferable, but is not absolutely required in all embodiments of the invention. In general, the tail structure will be present when multiple SNPs are amplified at the same time. For this reason, SNPs to be multiplexed will use the same forward and reverse tails. The principle that guides the design of the tails follows that of conventional PCR primer design, i.e. it requires the tails to have a sufficient number of nucleotides to achieve specific and robust PCR. In general, the length of the tail would be 4 to 45 bases, preferably 8 to 30 bases.

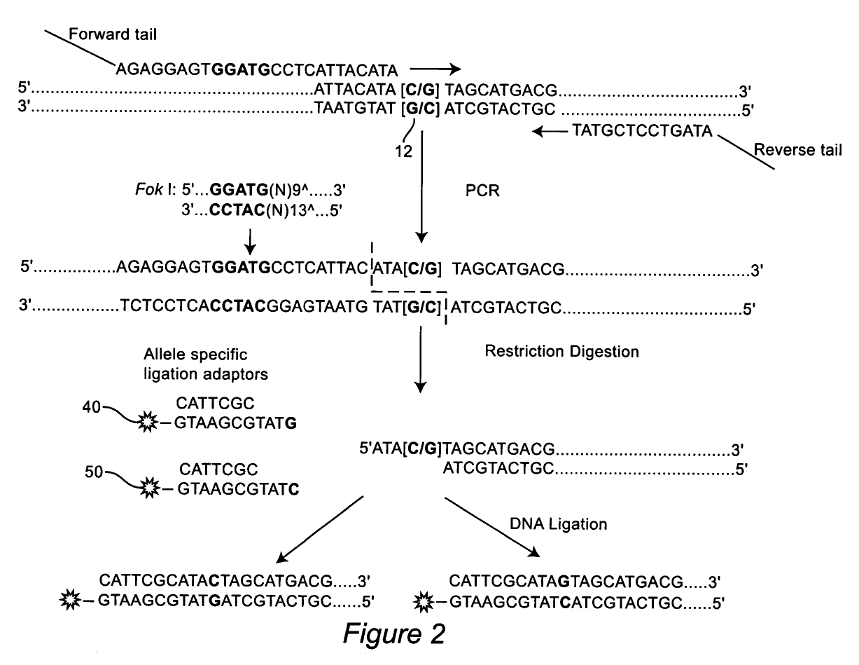

A second embodiment of the method of the present invention is represented schematically in FIG. 2. In FIG. 2, all the elements are identical to those of FIG. 1 except that after restriction digest of the PCR products, the nucleotide that is complementary to the SNP is joined to the digested PCR product as part of an allele-specific ligation adaptor sequence. The allele-specific ligation adaptor sequence 40 for the C allele of SNP 11 includes complementary nucleotide G at its 5′ end, and is differentially labeled with a detectable label. The allele-specific ligation adaptor sequence 50 for the G allele of SNP 11 includes complementary nucleotide C at its 5′ end, and is also differentially labeled, but with a different detectable label. The RE digestion products and the ligation adaptors undergo a DNA ligation reaction in which the adaptors are joined to the digestion products, creating allele-specific products that can be detected and distinguished from one another by a DNA sequencer, thus establishing the genotype of the SNP in the samples that are analyzed.

Thus, using this restriction enzyme mediated method, a series of PCR products that vary in size, and contain only one restriction enzyme recognition site, is created. This allows many allele-specific products to be loaded in a single capillary/lane, as demonstrated by the simultaneously typing of multiple SNPs for 44 DNA samples described in the Examples section below. By multiplexing PCR and pooling multiplexed reactions together, this method has the potential to score about 50 to about 100 or more SNPs/capillary/run if, for example, the sizes of PCR products are designed to vary at every 5 to 10 bases within a 100 to 600 base range. However, those of skill in the art will recognize that fewer SNPs can be analyzed at one time if desired. In general, the sizes of the PCR products for each SNP will differ from the size of PCR products of all other SNPs by at least about one nucleotide, and preferably by at least about 5 to 10 or more nucleotides.

This design enables the generation of a unique size of PCR product for each SNP, and unique labeling of each allele of an SNP, paving the road for high-level multiplex SNP typing by DNA sequencers. The technique overcomes the limitation of primer length and allows the entire size range of DNA sequencers to be used for genotyping. Using this method, the capacity of multiplexing is increased significantly, and the cost of operation is reduced.

In one embodiment of the invention, single base extension (SBE) is used in the joining step of the method. In this embodiment, dye-terminator ddNTPs may be used for the SBE reaction. In a preferred embodiment of the invention, the ddNTPs are labeled with a fluorescent label, examples of which include but are not limited to fluorescein-ddNTPs, TAMRA-ddNTPs, ROX-ddNTPs, R110-ddNTPs, R6G-ddNTPs, Cy3-ddNTPs, Cy5-ddNTPs and Texas Red-ddNTPs. In other embodiments of the invention, allele-specific ligation adaptors are employed. These ligation adaptors are also differentially labeled, for example, by Fluorescein, Rhodamine, TAMRA, ROX, R110, R6G, Cy3, Cy5 and Texas Red. By “differentially labeled” we mean that one label corresponds to one of the four potential nucleotides A, T, C or G. Some SNP may not vary by all possible nucleotides in all possible combinations. Nevertheless, all variations can be detected by the methods of the present invention, when differential labeling of the complementary nucleotide (as is well-known to those of skill in the art) is employed.

In one embodiment of the invention, the RE is a type IIS RE and leaves a 5′-overhang after cutting at the RE cleavage site. However, those of skill in the art will recognize that other enzymes that cut at a distance from their recognition site are also available for use in the present invention. In this case, a 3′-overhang is produced upon RE cleavage, and an allele specific adaptor with a 3′ overhang structure can be ligated to the digested PCR product. This embodiment uses the principle of oligonucleotide ligation assay²¹. However, the present invention differs from the oligonucleotide ligation assay is that the former uses type IIS REs to create sticky ends (either 5′ or 3′ overhang structure) that are allele-specific, whereas the latter uses synthesized short oligonucleotides to form a nicking structure at the ligation site. In general, both embodiments of the present invention (creation of 5′ and creation of 3′ overhangs) are similar, and the primer with the recognition site may be located on either the sense or antisense strand, with the second primer located on the opposing strand. However, for the embodiment in which a 3′ overhang is created, the directionality of the overhang and its components as shown in the Figures would be reversed, (e.g. the innermost nucleotide of the overhang would be the innermost 5′ nucleotide, i.e. the first nucleotide of the overhang in the 5′ to 3′ direction.

One advantage of the present invention is the ability to genotype multiple SNPs in the same reaction. Preferably, from 1 to about 100 SNPs can be genotyped at one time, and most preferably from about 5 to about 35 SNPs can be genotyped in a single reaction. In some embodiments of the invention, the reactions of amplification, restriction enzyme digestion, and joining of the complementary nucleotide (e.g. by extension or ligation) are carried out sequentially as separate reactions. This may be accomplished by carrying out the reactions in an isolated manner in separate reaction vessels, in which case the products of each reaction are transferred to a new reaction vessel for the next reaction, and additional reactive agents for the next reaction (e.g. a restriction enzyme) are added. Alternatively, the reaction components can be kept in a single tube and the agents necessary for carrying out a subsequent reaction can be added after a time sufficient to complete a previous reaction.

According to the method of the present invention, an instrument that is capable of determining the sequential order of nucleotides of a DNA fragment is utilized to detect the allele-specific products that are produced by the method. In a preferred embodiment of the invention, the instrument is a DNA sequencer. However, those of skill in the art will recognize that other techniques/instruments are available that are suitable for use in the method, examples of which include but are not limited to microarrays, micro chips, micro beads, and various micro fluidics devices. Still other detection platforms that may be employed in the invention include but are not limited to mass spectrometry, fluorescence resonance energy transfer, fluorescence polarization and melting temperature analysis.

### EXAMPLES

### Materials and Methods

DNA Samples

Human genomic DNAs were obtained from Coriell Institute (Camden, N.J.). The sample panel consisted of 44 individuals. The working concentration was 10 ng/μl.

PCR Primer Design

The forward primer was engineered to contain a type II RE recognition site at a specific position of the primer so that the restriction enzyme could cut the DNA fragment immediately upstream of the SNP site (in 5′ to 3′ direction). For example, the recognition sequence, GGATG, was placed 13 bases upstream of the targeted SNP site to generate a Fok I site, FIG. 1. Since the position of forward primer was fixed in this design, the reverse primer was positioned to produce a unique size for each SNP. When each SNP had a unique size, multiple SNPs could be stacked together for a sequencer run. Common tails (F: 5′-CGGTGCGCGTCGCTCAGG-3′ (SEQ ID NO: 1) for the forward primer, and R: 5′-TCCGATATCCCGGGTCGT-3′ (SEQ ID NO: 2) for the reverse primer) were added to the forward and reverse primers respectively to improve the performance of multiplex PCR. Eighteen randomly selected markers were designed for this study using the Primer 3 program, which is available at the “broad.mit.edu” website (21). Primers were obtained from Qiagen Cooperation (Alameda, CA). The marker information and primer sequences are listed in Table 2.

TABLE 2
SNP information and primer sequences

| | | | | Size^{a} |
|---|---|---|---|---|
| No | SNP | Primer Sequence | Allele | (bp) |
| 1 | rs1156853 | 5′-F-CAAGTTfCGGATGATAACCAGTA-3′(SEQ ID NO: 3) | [a/g] | 409 |
| | | 5′-R- AGAATTTTACCAGATCTCCAATGT-3′(SEQ ID NO: 4) | | |
| 2 | rs1345662 | 5′-F-CACTTAGAGCGGATGGTAATTATGTCT-3′(SEQ ID NO: 5) | [c/g] | 331 |
| | | 5′-R-GAGGGCAAGCCTCTCTATATC-3′(SEQ ID NO: 6) | | |
| 3 | rs1990001 | 5′-F-TCCAGGGGATGCATGTCCTGTTC-3′(SEQ ID NO: 7) | [a/g] | 171 |
| | | 5′-R-CCTTTCCCTGGCCTAGTACAG-3′(SEQ ID NO: 8) | | |
| 4 | rs257926 | 5′-F-TTCAACCGGATGCCAACTGAGCAC-3′(SEQ ID NO: 9) | [a/g] | 180 |
| | | 5′-R-TCCTGAAGGGATGAGTTCC-3′(SEQ ID NO: 10) | | |
| 5 | rs1864922 | 5′-F-ACCCGGATGCAACAGTCACC-3′(SEQ ID NO: 11) | [c/t] | 245 |
| | | 5′-R-TGCAAGAATTGAGCTTTAATA-3′(SEQ ID NO: 12) | | |
| 6 | rs246943 | 5′-F-CCTTATTTAGGGGATGTACAAACACTT-3′(SEQ ID NO: 16) | [c/t] | 143 |
| | | 5′-R-ACGCCCGGCAAGATTCAT-3′(SEQ ID NO: 14) | | |
| 7 | rs246945 | 5′-F-AGAGGAGTGGATGCCTCTAATGTT-3′(SEQ ID NO: 15) | [c/g] | 273 |
| | | 5′-R-GGACACGCAGAATGGGAGA-3′(SEQ ID NO: 16) | | |
| 8 | rs149445 | 5′-F-ATGAAAAGGATGGAGTCACTG-3′(SEQ ID NO: 17) | [c/t]467 | |
| | | 5′-R-AAATACATCTAACCATATTTTAAGAG-3′(SEQ ID NO: 18) | | |
| 9 | rs1560636 | 5′-F-AATGAAAAGGATGGAGTCACTG-3′(SEQ ID NO: 19) | [a/g] | 393 |
| | | 5′-R-ACCCCAGGAAAGGACAAAACAA-3′(SEQ ID NO: 20) | | |
| 10 | rs1422318 | 5′-F-AGTTCTTGGGATGAAGGAAAT-3′(SEQ ID NO: 21) | [a/g]490 | |
| | | 5′-R-CATTCCATGATATAATCTTTGTG-3′(SEQ ID NO: 22) | | |
| 11 | rs974495 | 5′-F-GTTGATGGGATGGTTAGAAAAAG-3′(SEQ ID NO: 23) | [a/g] | 160 |
| | | 5′-R-ACAACACAAGGTAGTTTCACG-3′(SEQ ID NO: 24) | | |
| 12 | rs298095 | 5′-F-CAGGTAGGATGGGGCTTTGTGTA-3′(SEQ ID NO: 25) | [a/t]525 | |
| | | 5′-R-TCTCTAACATACCTATCAAGTCTA-3′(SEQ ID NO: 26) | | |
| 13 | rs2109857 | 5′-F-TCCTGGGGATGGAAATAAGGAC-3′(SEQ ID NO: 27) | [a/g] | 267 |
| | | 5′-R-AGCGGAAACTGCCTTAGCTG-3′(SEQ ID NO: 28) | | |
| 14 | rs27563 | 5′-F-TGCTGGGATGCATTTTGATGTT-3′(SEQ ID NO: 29) | [a/t] | 209 |
| | | 5′-R-CCCACACAAGGGATTGAAA-3′(SEQ ID NO: 30) | | |
| 15 | rs2045628 | 5′-F-AGTATAACAGGATGGAAAGAGCTG-3′(SEQ ID NO: 31) | [a/g] | 243 |
| | | 5′-R-ATTCCTATTCTTGAAACCTCTGG-3′(SEQ ID NO: 32) | | |
| 16 | rs2041189 | 5′-F-TGCAAATCGGATGCCTCTAGC-3′(SEQ ID NO: 33) | [c/g] | 133 |
| | | 5′-R-TTCTACTTTTATTCCATCATTTGC-3′(SEQ ID NO: 34) | | |
| 17 | rs1609850 | 5′-F-TTGAGACGGATGTGACTAACACTG-3′(SEQ ID NO: 35) | [a/t] | 328 |
| | | 5′-R-CCAGGTAATGAATAATGTGAGGT-3′(SEQ ID NO: 36) | | |
| 18 | rs27562 | 5′-F-AGTTTACGGATGATTTAGGTCTCC-3′(SEQ ID NO: 37) | [g/c]223 | |
| | | 5′-R-GCAATTGTAAGATTCAGGGAAG-3′(SEQ ID NO: 38) | | |

F, the forward common tail: 5′-CGGTGCGCGTCGCTCAGG (SEQ ID NO: 1);
R, the reverse common tail: 5′-TCCGATATCCCGGGTCGT (SEQ ID NO: 2);
a, the size of the amplicons after RE digestion.

Some other REs that could be used for REM-SBE technique were listed above in Table 1.

Multiplex PCR

The optimization of multiplex PCR was implemented by a stepwise procedure. We first tested primer efficiency and specificity for each SNP individually using a three-step standard PCR protocol (94° C., 30 sec; 55° C. 45 sec; 65° C. 1 min, 35 cycles). We then pooled 4-5 markers that had different amplicon size and showed similar efficiency for multiplexing. Multiplex PCRs were performed using a two-step PCR protocol in 20 μl of reaction volume. For the first step, multiplex PCR was performed for 15 cycles with a reaction mixture containing 20 mM Tris-HCl (pH 8.4), 2.5 mM MgCl₂, 50 mM KCl, 6 mM (NH₄)₂SO₄, 30 nM of each primer (totally 10 primers for a 5-plex combination setup), 250 μM dNTPs (Invitrogen, Carlsbad, Calif.), 40 ng of DNA and 1 U of HotMaster Taq DNA polymerase (Eppendorf, Hamburg, Germany). After the first step, the PCR was paused to add a mixture of 5 μl containing 1 U of HotMaster Taq polymerase, 500 nM of each tail primer and 500 μM dNTPs. The reaction was resumed for 25 more cycles. Two optimized programs were used for multiplexing PCR. In program A, the first step consisted of 15 cycles of 95° C. for 30 sec, 58° C. for 5 sec, ramping down from 58 to 48° C. at 0.1° C./sec and 72° C. for 1 min. The second step used 25 cycles of 95° C. for 30 sec, 60° C. for 1 min and 72° C. for 1 min. In program B, the first step used 15 cycles of 95° C. for 30 sec, 60° C. for 45 sec with temperature decrement at −1° C./cycle and 72° C. for 1 min. The second step for program B was the same as in program A.

For visualization, PCR products were stained with SYBR Green (Molecular Probes, Eugene, Oreg.), stayed at room temperature for 20 min, then separated by electrophoresis on 2% Argrose Gel (Bio-Rad Laboratories, Hercules, Calif.). For any given gel analysis, the same amount of PCR products (5 μl) was loaded to each lane.

Restriction Digestion and SBE

After PCR amplification, 15 μl of PCR products were incubated with 2 U of shrimp alkaline phosphatase (Roche, Indianapolis, Ind.), and 4 U of Fok I RE (New England Biolabs, Beverly, Mass.) for 6 hours at 37° C. to digest unincorporated nucleotides and to cut the amplicons at the designed position. The enzymes were then inactivated by heating for 15 min at 85° C.

The restriction digested PCR products were labeled with SBE reaction using fluorescent terminator nucleotides. The SBE reaction contained 10 μl of digested PCR products, 2 μl of 10× sequencing buffer, I U of Taq DNA polymerase (New England Biolabs, Beverly, Mass.) and a mixture of fluorescent terminator nucleotides (5-(and -6)-carboxytetramethylrhodamine (TAMRA)-ddATP, TAMRA-ddCTP, rhodamine 110 (R110)-ddGTP, R110-ddUTP, 40 nM each) (Perkin Elmer, Boston, Mass.). The mixture of terminators was designed to use only two fluorescent dyes. When an A/C or G/T polymorphism was tested, R 110-ddCTP and TAMR-ddG would be used. This design was to simplifying color matrix correction. Distilled water was added to make a total of 20 μl reaction volume, and the mixture was incubated for 1 hour at 74° C.

Capillary Electrophoresis

Following the incubation, SBE reactions were diluted to 35 μl and purified by column filtration using a Performa 96-well plate (Edge Biosystems, Gaithersburg, Md.) following the manufacturer's instruction. One microliter of the filtered PCR product was resuspended in 9 μl deionized formamide with 0.1 μl of ILS-600 DNA size standard (Promega, Madison, Wis.). The fragments were then separated and identified by the SpectruMedix capillary sequencer SCE9610 (SpectruMedix LLC, State College, Pa.) using these conditions: sample injection at 3.0 KV for 120 sec; data acquisition at 1.0 KV for 120 min. Electrophoresis was performed using sequencing gel from SpectruMedix in TBE buffer (0.09 M Tris, 0.09 M boric acid, pH 8.0, 0.002 M EDTA). The GenoSpectrum software (SpectruMedix) was used to analyze the electropharogram.

Results

Multiplex PCR

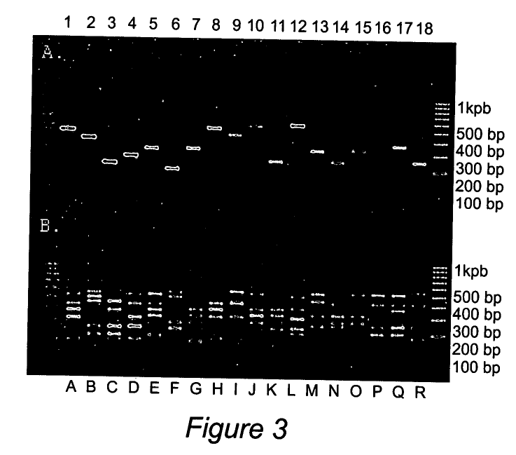

The goals were to use a DNA sequencer to increase throughput and reduce cost. To accomplish the goals, multiplexing was a necessity. A robust multiplex PCR protocol was an essential part of the SNP typing protocol. A two-step procedure was used to optimize multiplex PCR. Since the inclusion of a Foki site in the forward PCR primers could have a maximum of 5 base mismatches, single marker PCR was performed to examine the specificity and efficiency. As shown in FIG. 3A, single marker PCRs worked well for all 18 markers despite some variations in amplification efficiency. None of the markers had non-specific product. Interestingly, those markers that had lower efficiency in single marker PCR were not necessarily weaker in a multiplex setup (such as marker 10 in combination E and marker 16 in combination G, FIG. 3B). It was not clear whether the variation in efficiency was caused by the marker itself or the mismatch introduced in the forward PCR primers.

A minimal concentration of genomic DNA (≧2 ng/μl) for successful multiplex PCRs was observed. When the concentration of genomic DNA was lower than that, it would lead to insufficient amplification for some markers. While most 5-plex combinations of the 18 markers could be successfully amplified, the competition between primers caused some uneven amplification in some combinations. In a few combinations, some non-specific products with a size more than 1 kb were observed. The uniformity and specificity of multiplexed amplicons could be adjusted by using different PCR programs. The touchdown program, program B, did not generate any non-specific bands in any of the combinations tested. However, it had some difficulties in producing even PCR products in some of the 5-plex combinations. The ramping program, program A, on the other hand, had better uniformity for PCR products in the 5-plex sets (data not shown).

The 2-step PCR procedures allowed relatively even amplification of all multiplexed amplicons. The primer concentrations in the first step was found to be critical for successful multiplexing. In testing, the range of concentration was between 20-40 nM for each primer. When the concentration was too low, some amplicons in the multiplex would not be seen; on the other hand, higher concentration normally led to uneven amplifications of the amplicons. The use of the two-domain primers, as observed by others²², had effectively improved the multiplex.

Alkaline Phosphatase and Restriction Enzyme Digestions

After PCR, it was necessary to inactivate excess dNTPs and to create an extendable end at the targeted polymorphic site. These tasks were accomplished by digestions of shrimp alkaline phosphatase and type II RE. When the protocol was first tested, the two enzyme digestions were performed separately. In order to make the protocol more efficient, two combined reactions were tested. Side by side comparisons indicated that shrimp alkaline phosphatase and Fok I endonuclease did not interfere with each other when they were used together (data not shown). Combined or separated, the Fok I RE cut the DNA fragments precisely at the designed position for all 18 markers tested. The restriction digestion produced two DNA fragments for each marker and both fragments had a 5′-overhang structure that could be extended by DNA polymerase. For each marker, the smaller fragment of endonuclease digestion, which contained the enzyme recognition site, was a fragment of 40-50 bp (forward primer plus forward tail) and could not be easily distinguished between the markers. But for the larger fragments, they were designed to have a different size for each amplicon so that they could be resolved on a capillary sequencer.

When several 5-plex PCRs were pooled together for the digestions, the time of the digestions was extended from 6 to 8 hours. The amount of enzymes was kept the same. When three 5-plex PCRs were pooled for the digestions, identical results were obtained as those by individual PCRs (data not shown). Additional reactions were not pooled because only 18 markers were tested, and 15 of them were put into 3 multiplex reactions. Substantially more reactions could be pooled.

SBE and Genotyping Scoring

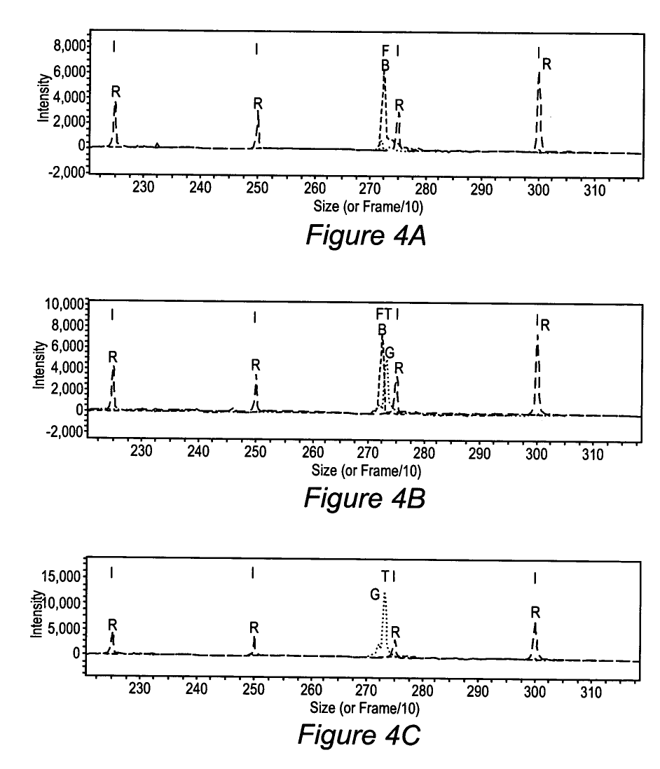

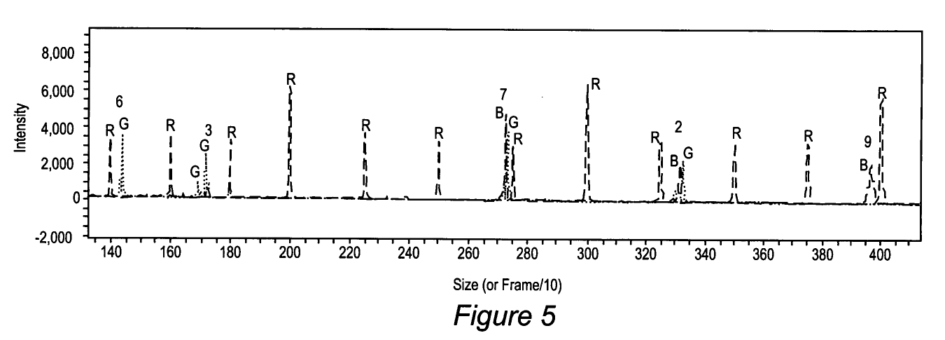

After the digestions, both the shrimp alkaline phashatase and Fok I endonuclease were inactivated by heating at 85° C. for 15 min. SBE was performed using Taq DNA polymerase and fluorescent terminators corresponding to the polymorphisms. Because the Fok I digestion created a 5′-overhang structure at the polymorphic site, there was no need to use any extension primer. DNA polymerases extended the ends of the restriction digestion and produced labeled, allele-specific DNA fragments. The labeled products could be easily separated and identified by DNA sequencers. The SBE was much more efficient when it was performed at elevated temperature because the sticky ends of Fok I digestion could anneal together at room temperature and reduced extension efficiency. This was the primary reason why a thermal stable DNA polymerase was used for the extension. A typical result is shown in FIG. 4. A homozygous sample had a single peak with one color and the color of the peak represented the allele. As seen in FIG. 4, panel A had a single peak F of 273 bases (labelled “B” for “blue”) as expected for marker rs246945, indicating that the sample was a homozygote for allele C. Similarly, a single peak T (labelled “G” for “green”) was seen in panel C of the figure, representing a homozygote for the G allele. For the heterozygote, two peaks “F” and “T” were seen (panel B, FIG. 4, where one is labeled “B” for blue and the other is labeled “G” for green). The peaks were often offset by a few data points. This was because each fluorescence group had a distinct mobility and the high resolution of CE was able to separate one from another. Even the same fluorophore could be separated when it was linked to primers of same length and sequence except the polymorphic base at 3′ end²³. The sensitivity of CE, therefore, was very helpful in identifying the heterozygous samples: they always had two peaks of different colors and the two peaks were offset by a few data points. The peak heights of the two peaks were approximately the same. While the peak height ratio changed between SNPs, it was constant for a given marker because the efficiency of incorporation of dye-terminators by Taq DNA polymerase was constant for a given sequence context^{24,25}. When several SNPs were multiplexed together, the peak high pattern of individual SNPs did not change (comparing the heterozygous in FIG. 4 with marker 7 in FIG. 5, which was the same SNP rs246945 run by itself or multiplexed with other SNPs).

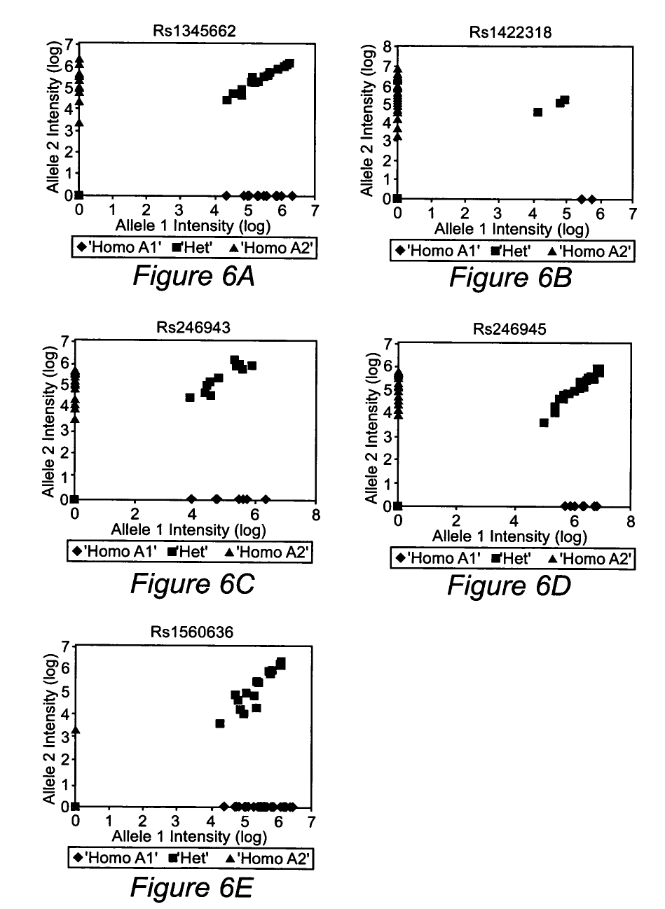

To verify the accuracy and efficiency of the protocol, 44 DNA samples were typed for 5 SNPs in a single 5-plex PCR. After CE, the color and size of peaks along with peak height and peak area were exported from GenoSpectrum, the genotyping software from our sequencer vendor SpectruMedix. Genotypes were scored by a Microsoft Excel template²⁷ implementing these criteria: i) product size was within the range of ±1.5 bases of expected size; ii) if the peak height ratio of allele 1/allele 2 was ≧10, the sample would be scored as homo allele 1; if the ratio was ≧0.1, the sample would be scored as homo allele 2; iii) when the peak height ratios were between 0.1 and 10, genotypes would be scored by a cluster algorithm based on Euclidean distances. The results of the 5-plex reaction is shown in FIG. 6. The genotypes scored from the 5-plex setup were a 100% match with the genotypes scored from single marker reaction and were in complete concordance with the genotypes obtained from a different technology^{28,29}.

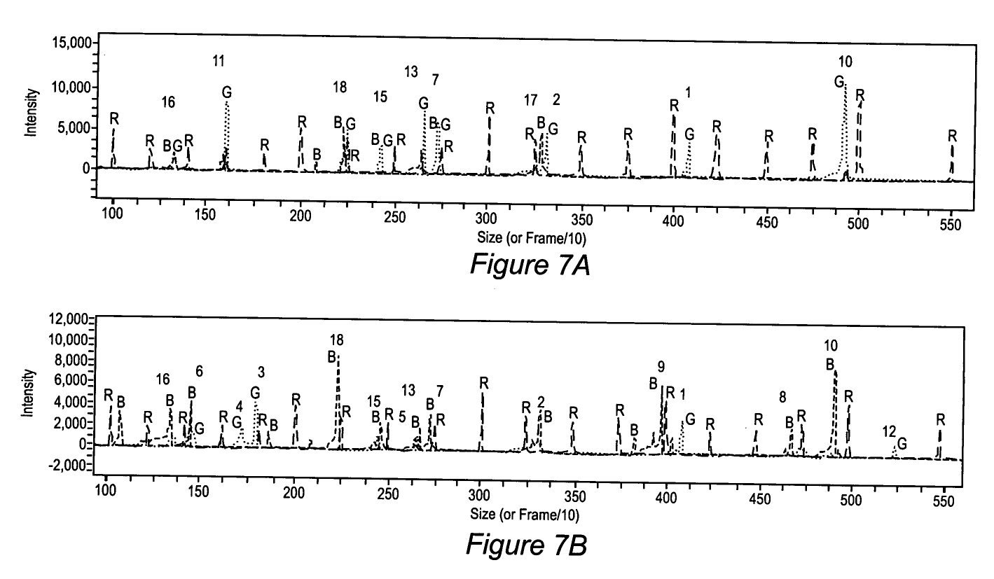

To be more efficient and cost-effective, it was desirable to pool several multiplex PCRs together. In the protocol, there were several stages at which reactions could be pooled. Reactions could be pooled after PCR, or pooled after phosphatase and endonuclease digestion, or pooled after gel filtration before sequencer run. Obviously, the earlier the reactions were pooled in the protocol, the more efficient the procedure would be. Multiplexing more SNPs in a PCR was tried and it was found that it was significantly more difficult when more than 5 SNPs were multiplexed. Therefore, multiplexing 5 SNPs in PCR was settled on. Several multiplexed PCRs were then pooled together for the phosphatase and endonuclease digestion. FIG. 7 shows some results of the pooling tests. Panel A was an experiment that pooled two 5-plex PCRs for the phosphatase and endonuclease digestion. Ten SNPs were clearly typed in a single capillary. Panel B was an experiment that pooled three 5-plex PCRs. All 15 markers were separated and typed. The genotypes scored from the pooled samples matched with those in the single 5-plex setup, indicating that the pooling of several reactions did not compromise the phosphatase and endonuclease digestion and did not sacrifice genotype quality.

Discussion

This example demonstrates the principles of a new SNP-typing method that uses type II restriction enzymes and DNA sequencers. The example shows that a recognition site can be engineered in one of the PCR primers and the mismatches introduced by the recognition site do not compromise the efficiency and specificity of PCR. The data further demonstrate that an extendable 5′-overhang structure is produced precisely immediately before targeted SNP sites and allele-specific products are produced by SBE reactions. The quality and accuracy of the method are illustrated by typing 5 SNPs simultaneously in a single PCR for 44 subjects.

In the protocol, the efficiency is increased by multiplexing PCR and by pooling several multiplexed reactions for a sequencer run. For multiplex PCR, a two-domain primer design that has a target-specific domain at the 3′ end and a common tag at the 5′ end was used. The two-staged procedure works well for multiplexing 4-5 markers. In the 18 SNPs used in this study, 5 SNPs were randomly selected to multiplex, and most of them worked well on the first try (FIG. 3 shows some of these results). In addition to the two-staged procedure and two-domain primer design, the primer concentration used in the first reaction and the mismatches introduced by the Fok I site in the primers contribute to the success of multiplex PCR. Pooling of several multiplexed reactions for enzyme digestions, cleanup and sequencer run is a key to reduce overall cost of genotyping because these are the most time-consuming and expensive steps in the protocol. The more pooling, the lower the cost would be. If ten 5-plex reactions are pooled, 50 SNPs could be typed in a capillary. The throughput would be significant and the cost would be competitive.

The use of a RE to produce extendable ends at the polymorphic sites makes it difficult to type those SNPs that are located close to the same restriction recognition sequence used in the PCR primers. This is because if there is another RE site close to the SNP, RE digestion of the PCR product will produce a second sticky end, which would interfere with the allele-specific single base extension or ligation reaction intended for the sticky end at the SNP site. However, this weakness can be overcome by using a different RE. Some of the REs that can be used in this invention include Fok I, Bbv I, BtgZ I and Bce AI (Table 1). All these enzymes have at least 8 basepairs between the cutting site and recognition sequence, and this is sufficient to allow specific and robust PCR, as demonstrated by Fok I whose distance is 9 basepairs between the cutting site and recognition sequence (FIG. 1). If one enzyme does not work for a given SNP, a different one can be used. Due to the limitation of distance between recognition and cutting sites of the restriction enzymes, PCR primer design may be restricted to some extent. Since there are two orientations to design a PCR primer for a given SNP, this is manageable.

In conclusion, this example shows the development of a SNP typing method that combines the accuracy of SBE reaction and the sensitivity of CE. SBE is one of the best biochemistries for SNP typing and most commercially available techniques today use this same biochemistry. DNA sequencers and other CE platforms have been proven for use in high throughput and automation. The primary limitation to use CE efficiently is the creation of a set of allele specific products with different lengths. The present invention overcomes this barrier by taking advantage of type II restriction enzymes and engineering an enzyme recognition site in one of the PCR primers. This design makes it possible to obtain allele-specific products. By varying the size of PCR products purposefully, it is possible to stack many SNPs in a capillary and use the resolution power of CE efficiently. As a result, DNA sequencers can be used for SNP typing efficiently and economically.

While the invention has been described in terms of its preferred embodiments, those skilled in the art will recognize that the invention can be practiced with modification within the spirit and scope of the appended claims. Accordingly, the present invention should not be limited to the embodiments as described above, but should further include all modifications and equivalents thereof within the spirit and scope of the description provided herein.

### REFERENCES

1 Sachidanandam R, Weissman D, Schmidt S C, Kakol J M, Stein L D, Marth G, Sherry S, Mullikin J C, Mortimore B J, Willey D L, Hunt S E, Cole C G, Coggill P C, Rice C M, Ning Z, Rogers J, Bentley D R, Kwok P Y, Mardis ER, Yeh RT, Schultz B, Cook L, Davenport R, Dante M, Fulton L, Hillier L, Waterston R H, McPherson J D, Gilman B, Schaffner S, Van Etten W J, Reich D, Higgins J, Daly M J, Blumenstiel B, Baldwin J, Stange-Thomann N, Zody M C, Linton L, Lander E S, Atshuler D. A map of human genome sequence variation containing 1.42 million single nucleotide polymorphisms. Nature. 2001; 409: 928-933.
2 Venter J C, Adams M D, Myers EW, Li P W, Mural R J, Sutton GG, Smith H O, Yandell M, Evans C A, Holt R A, Gocayne J D, Amanatides P, Ballew R M, Huson D H, Wortman J R, Zhang Q, Kodira C D, Zheng X H, Chen L, Skupski M, Subramanian G, Thomas P D, Zhang J, Gabor Miklos G L, Nelson C, Broder S, Clark A G, Nadeau J, McKusick V A, Zinder N, Levine A J, Roberts R J, Simon M, Slayman C, Hunkapiller M, Bolanos R, Delcher A, Dew I, Fasulo D, Flanigan M, Florea L, Halpern A, Hannenhalli S, Kravitz S, Levy S, Mobarry C, Reinert K, Remington K, Abu-Threideh J, Beasley E, Biddick K, Bonazzi V, Brandon R, Cargill M, Chandramouliswaran I, Charlab R, Chaturvedi K, Deng Z, Di F, V, Dunn P, Eilbeck K, Evangelista C, Gabrielian A E, Gan W, Ge W, Gong F, Gu Z, Guan P, Heiman T J, Higgins M E, Ji R R, Ke Z, Ketchum K A, Lai Z, Lei Y, Li Z, Li J, Liang Y, Lin X, Lu F, Merkulov G V, Milshina N, Moore H M, Naik A K, Narayan V A, Neelam B, Nusskem D, Rusch D B, Salzberg S, Shao W, Shue B, Sun J, Wang Z, Wang A, Wang X, Wang J, Wei M, Wides R, Xiao C, Yan C, Yao A, Ye J, Zhan M, Zhang W, Zhang H, Zhao Q, Zheng L, Zhong F, Zhong W, Zhu S, Zhao S, Gilbert D, Baumhueter S, Spier G, Carter C, Cravchik A, Woodage T, Ali F, An H, Awe A, Baldwin D, Baden H, Barnstead M, Barrow I, Beeson K, Busam D, Carver A, Center A, Cheng M L, Curry L, Danaher S, Davenport L, Desilets R, Dietz S, Dodson K, Doup L, Ferriera S, Garg N, Gluecksmann A, Hart B, Haynes J, Haynes C, Heiner C, Hladun S, Hostin D, Houck J, Howland T, Ibegwam C, Johnson J, Kalush F, Kline L, Koduru S, Love A, Mann F, May D, McCawley S, McIntosh T, McMullen I, Moy M, Moy L, Murphy B, Nelson K, Pfannkoch C, Pratts E, Puri V, Qureshi H, Reardon M, Rodriguez R, Rogers Y H, Romblad D, Ruhfel B, Scott R, Sitter C, Smallwood M, Stewart E, Strong R, Suh E, Thomas R, Tint N N, Tse S, Vech C, Wang G, Wetter J, Williams S, Williams M, Windsor S, Winn-Deen E, Wolfe K, Zaveri J, Zaveri K, Abril J F, Guigo R, Campbell M J, Sjolander K V, Karlak B, Kejariwal A, Mi H, Lazareva B, Hatton T, Narechania A, Diemer K, Muruganujan A, Guo N, Sato S, Bafna V, Istrail S, Lippert R, Schwartz R, Walenz B, Yooseph S, Allen D, Basu A, Baxendale J, Blick L, Caminha M, Carnes-Stine J, Caulk P, Chiang Y H, Coyne M, DahLke C, Mays A, Dombroski M, Donnelly M, Ely D, Esparham S, Fosler C, Gire H, Glanowski S, Glasser K, Glodek A, Gorokhov M, Graham K, Gropman B, Harris M, Heil J, Henderson S, Hoover J, Jennings D, Jordan C, Jordan J, Kasha J, Kagan L, Kraft C, Levitsky A, Lewis M, Liu X, Lopez J, Ma D, Majoros W, McDaniel J, Murphy S, Newman M, Nguyen T, Nguyen N, Nodell M. The sequence of the human genome. Science. 2001; 291: 1304-1351.
3 Kwok P Y. Methods for genotyping single nucleotide polymorphisms. Annu. Rev. Genomics Hum. Genet. 2001; 2: 235-258.
4 Chen X, Sullivan P F. Single nucleotide polymorphism genotyping: biochemistry, protocol, cost and throughput. Pharmacogenomics. J 2003; 3: 77-96.
5 Syvanen A C. Accessing genetic variation: Genotyping single nucleotide polymorphisms. Nat. Rev. Genet. 2001; 2: 930-942.
6 Fan J B, Chen X, Halushka M K, Bemo A, Huang X, Ryder T, Lipshutz R J, Lockhart D J, Chakravarti A. Parallel genotyping of human SNPs using generic high-density oligonucleotide tag arrays. Genome Res. 2000; 10: 853-860.
7 Armstrong B, Stewart M, Mazumder A. Suspension arrays for high throughput, multiplexed single nucleotide polymorphism genotyping. Cytometry. 2000; 40: 102-108.
8 Griffin T J, Smith L M. Single-nucleotide polymorphism analysis by MALDI-TOF mass spectrometry. Trends Biotechnol. 2000; 18: 77-84.
9 Bray M S, Boerwinkle E, Doris P A. High-throughput multiplex SNP genotyping with MALDI-TOF mass spectrometry: practice, problems and promise. Hum. Mutat. 2001; 17: 296-304.
10 Bell P A, Chaturvedi S, Gelfand C A, Huang C Y, Kochersperger M, Kopla R, Modica F, Pohl M, Varde S, Zhao R, Zhao X, Boyce-Jacino M T. SNPstream UHT: ultra-high throughput SNP genotyping for pharmacogenomics and drug discovery. Biotechniques. 2002; Suppl: 70-77.
11 Livak K J. Allelic discrimination using fluorogenic probes and the 5′ nuclease assay. Genet. Anal. 1999; 14: 143-149.
12 Ronaghi M. Pyrosequencing sheds light on DNA sequencing. Genome Res. 2001; 11: 3-11.
13 Chen X, Levine L, Kwok P Y. Fluorescence polarization in homogeneous nucleic acid analysis. Genome Res. 1999; 9: 492-498.
14 Heller C. Principles of DNA separation with capillary electrophoresis. Electrophoresis. 2001; 22: 629-643.
15 Schouten J P, McElgunn C J, Waaijer R, Zwijnenburg D, Diepvens F, Pals G. Relative quantification of 40 nucleic acid sequences by multiplex ligation-dependent probe amplification. Nucleic Acids Res. 2002; 30: e57-
16 Medintz I, Wong W W, Sensabaugh G, Mathies R A. High speed single nucleotide polymorphism typing of a hereditary haemochromatosis mutation with capillary array electrophoresis microplates. Electrophoresis. 2000; 21: 2352-2358.
17 Ayyadevara S, Thaden J J, Shmookler Reis R J. Discrimination of primer 3′-nucleotide mismatch by taq DNA polymerase during polymerase chain reaction. Anal. Biochem. 2000; 284: 11-18.
18 Liu W H, Kaur M, Makrigiorgos G M. Detection of hotspot mutations and polymorphisms using an enhanced PCR-RFLP approach. Hum. Mutat. 2003; 21: 535-541.
19 Ronai Z, Sasvari-Szekely M, Guttman A. Miniaturized SNP detection: quasi-solid-phase RFLP analysis. Biotechniques. 2003; 34: 1172-1173.
20 Pingoud A, Jeltsch A. Structure and function of type II restriction endonucleases. Nucleic Acids Res. 2001; 29: 3705-3727.
21 Chen X, Livak K J, Kwok P Y. A homogeneous, ligase-mediated DNA diagnostic test. Genome Res. 1998; 8: 549-556.
22 Rozen S, Skaletsky H. Primer3 on the WWW for general users and for biologist programmers. Methods Mol. Biol. 2000; 132: 365-386.
23 Shuber A P. Universal primer sequence for multiplex DNA amplification. 1999;
24 Matyas G, Giunta C, Steinmann B, Hossle J P, Hellwig R. Quantification of single nucleotide polymorphisms: a novel method that combines primer extension assay and capillary electrophoresis. Hum. Mutat. 2002; 19: 58-68.
25 Parker L T, Deng Q, Zakeri H, Carlson C, Nickerson D A, Kwok P Y. Peak height variations in automated sequencing of PCR products using Taq dye-terminator chemistry. Biotechniques. 1995; 19: 116-121.
26 Parker L T, Zakeri H, Deng Q, Spurgeon S, Kwok P Y, Nickerson D A. AmpliTaq DNA polymerase, FS dye-terminator sequencing: analysis of peak height patterns. Biotechniques. 1996; 21: 694-699.
27 van den Oord E J, Jiang Y, Riley B P, Kendler K S, Chen X. FP-TDI SNP scoring by manual and statistical procedures: a study of error rates and types. Biotechniques. 2003; 34: 610-20, 622.
28 Chen X, Levine L, Kwok P Y. Fluorescence polarization in homogeneous nucleic acid analysis. Genome Res. 1999; 9: 492-498.
29 Chen X. Fluorescence polarization for single nucleotide polymorphism genotyping. Comb. Chem. High Throughput. Screen. 2003; 6: 213-223.

1. A method of determining a genotype of a single nucleotide polymorphism (SNP), comprising the steps of
a. amplifying by polymerase chain reaction (PCR) a nucleotide sequence containing said SNP using a first primer and a second primer,
said first primer binding to a first strand of DNA containing said SNP and comprising
sequences identical to a recognition site of a restriction enzyme
a first DNA sequence homologous to nucleotide sequences immediately adjacent to said SNP; and
a tail comprising DNA sequences that are not homologous to sequences flanking said SNP;
said second primer binding to a second strand of DNA containing said SNP and comprising
DNA sequences homologous to nucleotide sequences flanking said SNP on said second strand; and
a tail comprising DNA sequences that are not homologous to said nucleotide sequence;
wherein said step of amplifying produces amplification products that contain said recognition site of a restriction enzyme and a cleavage site of said restriction enzyme, and wherein a terminal nucleotide of said cleavage site is one allele of said SNP;
b. cleaving said amplification product with said restriction enzyme, wherein said step of cleaving produces a cleaved amplification product with an overhang in which a first nucleotide of said overhang is one allele of said SNP;
c. joining a detectable complementary nucleotide to said one allele of said SNP to form an allele specific detectable product;
d. detecting said allele specific detectable product produced in said joining step; and
e. genotyping said SNP based on output from said detecting step.

2. The method of claim 1, wherein said first primer further comprises a second DNA sequence homologous to nucleotide sequences flanking but not immediately adjacent to said SNP, and wherein said sequences identical to a recognition site of a restriction enzyme are located between said first and second DNA sequences.

3. The method of claim 1, wherein said restriction enzyme is a type IIS restriction enzyme.

4. The method of claim 3 wherein said type IIS restriction enzyme is selected from the group consisting of BbvI, BceAI, BtgZI and FokI.

5. The method of claim 1, wherein said overhang is a 5′ overhang and said step ofjoining is carried out by single base extension with differentially labeled nucleotides.

6. The method of claim 5, wherein said differentially labeled nucleotides are fluorescent dye-terminator nucleotides.

7. The method of claim 1, wherein said overhang is a 3′ overhang and said step of joining is carried out by a method selected from the group consisting of DNA ligation using allele specific ligation adaptors and DNA hybridization using allele specific hybridization probes.

8. The method of claim 1, wherein said first primer is a forward primer and said second primer is a reverse primer.

9. The method of claim 1, wherein said second primer is a forward primer and said first primer is a reverse primer.

10. The method of claim 1, wherein said detecting step is performed with an instrument or technique selected from the group consisting of DNA sequencers, microarrays, microbeads, microchips, fluorescence resonance energy transfer, fluorescence polarization, melting temperature analysis, and mass spectrometry.

11. The method of claim 10 wherein said instrument or technique is a DNA sequencer.

12. The method of claim 10 wherein said instrument or technique is mass spectrometry.
